(19) United States
(12) Patent Application Publication
(10) Pub. No.: US 2013/0281845 A1
(43) Pub. Date: Oct. 24, 2013

(54) LIGHT EMITTING DIODE ENDOSCOPIC DEVICES FOR VISUALIZATION OF DISEASED TISSUE IN HUMANS AND ANIMALS

(71) Applicant: ONCOFLUOR, INC., Coronado, CA (US)

(72) Inventor: George A. Luiken, Coronado, CA (US)

(73) Assignee: Oncofluor, Inc.

(21) Appl. No.: 13/864,766

(22) Filed: Apr. 17, 2013

Related U.S. Application Data
(60) Provisional application No. 61/635,074, filed on Apr. 18, 2012.

(51) Int. Cl. A61B 5/00; A61B 1/04

(52) U.S. Cl. CPC A61B 5/0071; A61B 1/041
USPC 600/431; 600/109

(57) ABSTRACT

Endoscopic devices and methods for imaging and treating organs and tissues are described. The endoscopic devices described herein include flexible endoscopes, rigid endoscopes, and capsule endoscopes. The endoscopic device may comprise one or more cameras and one or more light sources. In some embodiments, the endoscopic device comprises at least one white light camera, at least one blue light camera, at least one white light source, and at least one blue light source. In some embodiments, fluorescent targeting constructs can be injected into the subject and bound to and/or taken up by a tumor or diseased tissue. Diseased tissue can be identified by viewing the fluorescence emanating from the fluorescent targeting constructs by illuminating an in vivo body part of the subject with light having at least one excitation wavelength in the range from 400 nm to about 510 nm.

### BACKGROUND OF THE INVENTION

### Field of the Invention

The present invention relates to endoscopic devices with digital image capture for viewing the state of a body cavity or an internal organ of a patient.

### SUMMARY OF THE INVENTION

Disclosed herein is an endoscopic device. In one embodiment, the endoscopic device comprises at least one white light source, at least one blue light source which emits light with a wavelength between 400 nm and 510 nm, a first camera, and a first filter capable of filtering light with a wavelength less than 515 nm.

Also disclosed is a method of detecting diseased tissue of a subject in need thereof. In one embodiment, the method comprises administering a diagnostically effective amount of a targeting construct to a subject, wherein the targeting construct is capable of specifically binding to and/or being taken up by the diseased tissue of the subject, and illuminating a body part of the subject with light having at least one excitation wavelength in the range from about 400 to about 510 nm, wherein the targeting construct fluoresces in response to the at least one excitation wavelength. In one embodiment, the method also comprises viewing fluorescence emanating from the targeting construct through a first camera and determining the location and/or surface area of the diseased tissue.

### BRIEF DESCRIPTION OF THE DRAWINGS

FIG. 1 depicts an embodiment of an endoscopic device.

FIG. 2 depicts an embodiment of a capsule endoscopic device.

### DETAILED DESCRIPTION OF THE INVENTION

As used herein, the term “monoclonal antibody” includes, but is not limited to, fully human antibodies, humanized antibodies, chimeric antibodies, whole antibodies, partial antibodies, Fab fragment antibodies, bispecific antibodies, diabodies, antibody fragments, etc.

As used herein, the term “fluorophore” means any non-toxic substance with excitation spectra in the visible light range (401-510 nm) and with emission spectra in the visible range (515-600 nm) with examples being fluorescein and fluorescein like derivatives, antibiotics (i.e. tetracycline), quinine, and quantum dots.

As used herein, the term “diseased tissue” includes, but is not limited to, cancer, endocrine adenomas, benign tumors with systemic effects.

In some embodiments, a method is disclosed which includes, (1) Diagnosis of a potentially resectable and surgically curable cancer; (2) Identification of surface or internal antigens on or within the tumor or other diseased cells; (3) Injection of fluorophore-tagged (and chemotherapy-tagged or chemotherapy/radioisotope-tagged anti-tumor antigen MAb; (4) Surgical resection of all visibly fluorescent tumor tissue (1-5 days after injection of the MAb); and (5) Adjuvant Therapy, including destruction of microscopic (and not visible) residual cancer cells through the attached fluorophore-tagged, plus chemotherapy or chemotherapy/radioisotope-tagged MAb.

Disclosed herein are endoscopic devices with digital image capture for viewing the state of a body cavity or an internal organ of a patient (human or animal) to allow accurate location and identification through tissue fluorescence and removal of diseased tissue. Endoscopic devices include flexible endoscopes (flexible such as in fiberoptic colonoscopes, bronchoscopes, culposcopes, etc.), rigid endoscopes (i.e. laparoscopes, sigmoidoscopes, thoracoscopes, culposcopes, cystoscopes, etc.) and capsule endoscopes (i.e. PillCam®, or Olympus Capsule endoscopes). Furthermore, endoscopic devices can have digital image capture devices mounted at the distal viewing end (or the end inserted into the body cavity) of the endoscopic devices. Diseased tissue could include cancer (of any organ), inflammation, hyper-functioning tissue (i.e. parathyroid adenomas, pituitary adenomas, adrenal adenomas, insulinomas, thyroid nodules and such). Endoscopic detection of diseased tissue can be through the visualization of the fluorophore-tagged tumor or tissue targeted monoclonal antibodies (MAbs or FlutaMabs) or fluorophore-tagged tissue or tumor-avid compounds (TACs). These fluorophore-tagged MAbs or TAcs can be injected into the human subject or animal to be examined one or more days prior to undertaking the examination to allow for binding of the fluorphore-tagged targeting construct to bind to the tumor or diseased tissue (see prior patents by George A. Luiken). In addition, at times these fluorophore-tagged MAbs or TACs may also have attached dual energy emission radio-isotopes (gamma and beta emitters, e.g. Iodine-131, and lutetium-177), or short range targeted alpha therapy (TAT) or alpha radio-immunotherapy (e.g. Lead-212). The short range of the energetic alpha emissions can be targeted directly to the diseased or tumor tissue or microscopic clusters of cells by delivery using fluorophore-tagged MAbs or fluorophore-tagged TACs. In some embodiments, the endoscopic device can use blue LED excitation light (400-510 nm) sources in addition to white LED light sources for illumination of the disease tissue of interest coupled with 515 nm filters for blocking the blue excitation light but allowing the green fluorescent emission light. In an embodiment, the endoscopic device incorporates radiation detection devices at the distal detection end of the endoscopic device. Wireless transmission of data and intra-subject geographic relocation of the disease tissue of interest using GPS type reference guidance can also be incorporated.

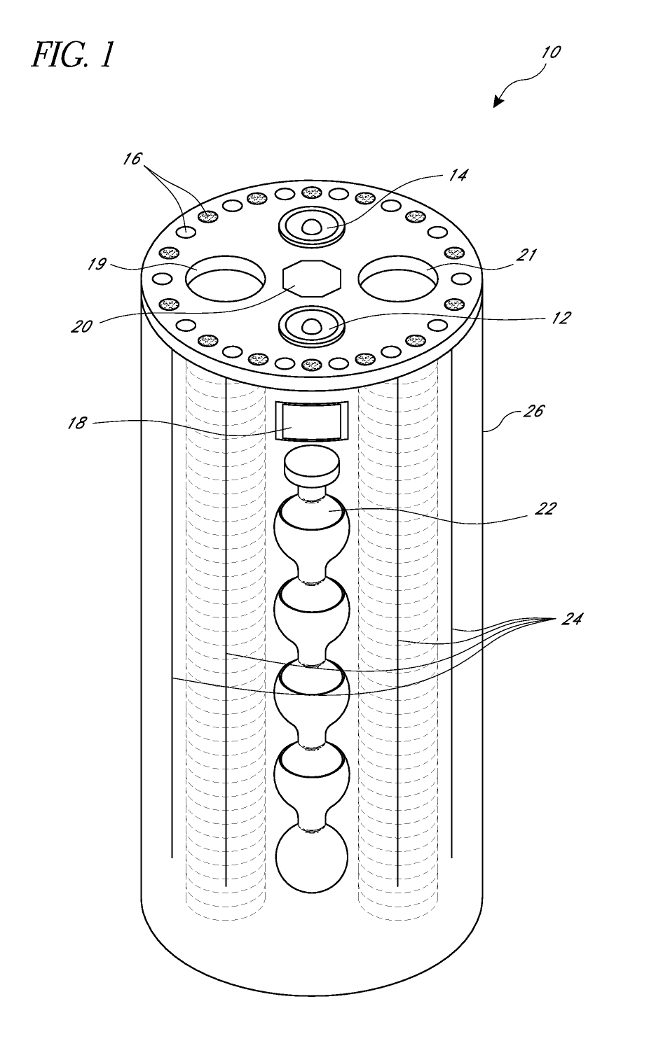

FIG. 1 depicts an embodiment of an endoscopic device 10. In some embodiments, the endoscopic device 10 comprises one or more imaging devices 12 and 14, which can be located at the distal end of the endoscopic device 10. In an embodiment, the imaging devices are one or more white light cameras 12 and one or more blue light cameras 14. In an embodiment, the endoscopic device 10 comprises two white light cameras 12 and two blue light cameras 14 which can display a three dimensional image to a user. In some embodiments, the endoscopic device 10 also comprises one or more light sources 16, which can also be located at the distal end of the endoscopic device 10. In an embodiment, the endoscopic device 10 comprises a ring of blue and white LED light sources. In an embodiment, the ring of blue and white LED sources alternates between blue and white LED lights. In some embodiments, the endoscopic device 10 includes one or more filters. For example, the endoscopic device 10 may include a yellow filter which may be located over the one or more blue light cameras 14. In some embodiments, the endoscopic device 10 also includes a radiation detection device 18, such as a Geiger counter, which may also be located near the distal end of the endoscopic device 10. In some embodiments, the endoscopic device 10 may also include one or more tubes 19. The one or more tubes 19 can be used for several purposes. For example, a tube 19 may be used as channel to collect samples to biopsy or to place an additional small rear facing camera. In some embodiments, the endoscopic device 10 may include a positioning device 20. For example, the positioning device 20 may include a geographic localization (GPS type) guidance chip which is capable of providing surgical trajectory and approach information. In an embodiment, the positioning device 20 is located near the distal end of the endoscopic device 10. In some embodiments, the endoscopic device 10 may also comprise one or more channels 21. The one or more channels 21 can be used for several purposes, such as fluid intrusion and aspiration.

In some embodiments, the endoscopic device 10 may include a structural device 22. For example, the endoscopic device 10 may include plastic interlocking units as shown in FIG. 1 which provide a flexible structure. The endoscopic device may also comprise one or more wires 24 which can be configured to be in electrical communication with any of the components of the endoscopic device 10. For example, wires may be in electrical communication with an external viewing device and one or more of the imaging devices 12 and 14. The wires 24 may also be used to flex the endoscope around turns. The endoscopic device 10 may also comprise an outer wall 26 which covers the inner components, such as the positioning device 20, the structural device 22, and/or the wires 24.

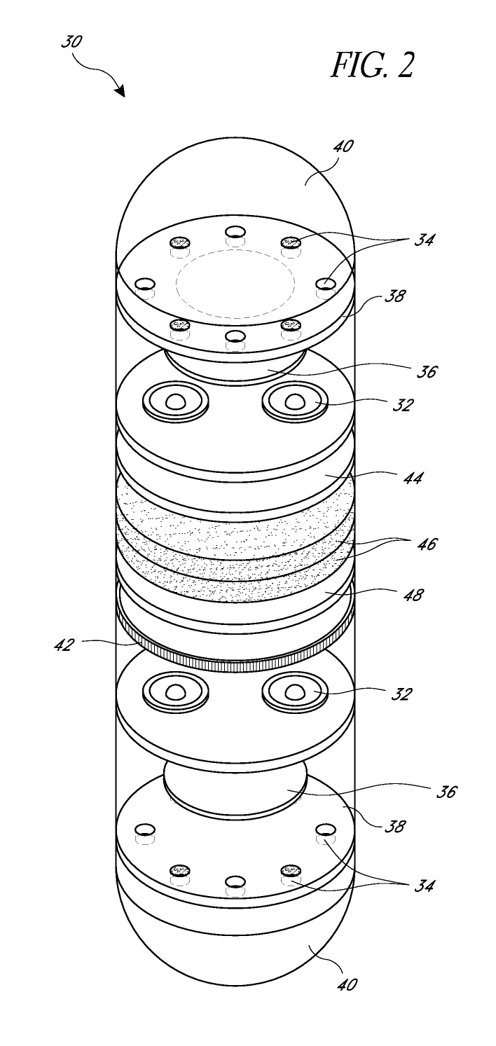

FIG. 2 depicts an embodiment of a capsule endoscopic device 30. In some embodiments, the capsule endoscopic device 30 may include one or more imaging devices 32. In some embodiments, the capsule endoscopic device 30 also comprises one or more light sources 34. For example, the capsule endoscopic device 30 may include one or more blue LED and white LED light sources 34. In some embodiments, the capsule endoscopic device 30 includes one or more lenses 36 and one or more lens holders 38. In some embodiments, the lenses 36 include one or more filters. For example, the lenses 36 may include a yellow filter. In some embodiments, the capsule endoscopic device 30 may include one or more optical domes 40 which provide a clear viewing path for the one or more imaging devices 32.

In some embodiments, the capsule endoscopic device 30 also includes a radiation detection device 42, such as a Geiger counter, which may also be located at the distal end of the endoscopic device 10. In some embodiments the capsule endoscopic device 30 also includes an antenna 44 capable of transmitting data, one or more batteries 46, and/or a transmitter 48.

In some embodiments, the endoscopic devices may be equipped with digital image capture devices (cameras) at the viewing end for imaging tissue with white light as well as with blue LED (400-510 nm, preferably 470-495 nm) light.

Multiple digital cameras (2 or more) may be used for viewing in 2 directions and in 3 dimensions (3-D).

Digital cameras may be of the small standard cell phone or micro-digital camera type or extremely small (Nano-Eye®) type for small diameter scopes.

One of the digital cameras may be equipped with a yellow filter (515 nm) or similar blocking filter to eliminate blue light emanating from the blue LED light source (400-510 nm) and the emission light emanating from the viewed fluorophore-tagged diseased tissue.

The diseased or tumor tissue within the body cavity or organ may be able to be localized and identified through the use of fluorescent-targeting constructs (fluorescent-tagged monoclonal antibodies (MAbs) or fluorescent-tagged tumor-avid or tissue-avid compounds (TACs) (said fluorophores having excitation (400-510 nm) and emission spectra (515-600 nm)) well within the visible range.

In addition the proximal end of each endoscopic device can also be fitted with micro-radiation detection devices (miniature Geiger counters) to detect tumor tissue at an interior body site (made possible by the radioisotope-labeled tissue targeting construct).

Excitation light at the distal or viewing end of the endoscopic devices can be extremely small white LEDs as well as blue LEDs (light-emitting diodes) (400-510 nm) capable of providing adequate light to view the internal organ and adequate light to excite the appropriate fluorophores.

Fluorophores used can be those with excitation spectra in the blue (400-510 nm) range and with emission spectra in the visible (515-600 nm) range.

The blue excitation (400-510 nm) light can be blocked from view through the use of a 515 nm filter mounted on the camera used for detection of fluorophore-tagged tissue.

In addition, the fluorophore-tagged tumor-avid or tissue-avid constructs (MAbs or TACs) may be combined with radio-isotopes with dual energy emission capabilities (beta and gamma emittors) for external and internal (endoscopic) nuclear scanning as well as providing therapeutic radiation to the diseased tissue when desired.

The endoscopic devices may also have embedded near the distal or viewing end, radiation detection devices (i.e. micro-Geiger counters) that are capable of detecting the radiation emitting from radio-isotopes, (said radio-isotopes being attached to the fluorophore-tagged tumor targeting constructs).

The endoscopic devices may be similar to standard externally manipulated rigid or flexible endoscopes (i.e. colonoscopes, bronchoscopes, gastroscopes, cystoscopes, arthroscopes, culposcopes, etc.) currently in use or currently available capsule endoscopes (i.e. PillCam®).

The endoscopic devices may also be equipped with wireless transmission capabilities for data capture external to the body cavity or organ being examined (image capture using white and/or blue LED (400-510 nm) light, radiation detection, and GPS type location capabilities of the image or radiation detected). This can provide the capability to re-locate an area of interest within the body cavity if needed at a later time.

Combining radio-isotopes to fluorophore-tagged tumor targeting constructs can provide 2 simultaneous methods for accurately identifying diseased or tumor tissue within a human or animal subject in need thereof. It can allow accurate localization and identification of tumor tissue prior to any surgical procedure using external nuclear scanning equipment as well as allowing localization through endoscopic detection of radio-isotopes and fluorescence attached to the tumor-targeting fluorescent constructs during an endoscopic or open surgical procedure.

The use of fluorophores with excitation (400-510 nm) and emission spectra (515-600 nm) within the visible light range can allow for direct viewing (without the aid of a capture device i.e. as a CCD) of the diseased tissue if the subject being examined should have the need for open field surgery at any time during the examination procedure. For example, this could occur if a patient undergoing colonoscopic resection of a colon cancer had a more extensive disease than originally thought and then required an open incision to complete the surgical procedure.

Power for white and blue LEDs of the endoscopic devices (flexible, rigid, or capsule) can be provided by wire from an external electrical source, or via batteries embedded in the distal end of the endoscopic device.

Image capture and recording of the digital data (images with white and blue LED lighting, radiation detection, and geographic positioning) can be provided wirelessly to standard smartphone, tablet device (i.e. iPad, Samsung tablet Kindle, etc.), laptop or desktop computer or television.

Streaming of image capture to smartphone, tablet (i.e. iPad), laptop or desktop computer and to remote locations can be provided by cell phone service provider.

Each camera may be capable of fish eye lens attachment to provide 180 degrees of visual field viewing.

Wireless localization devices placed at fixed locations on the body (i.e. pelvic symphysis, sternal notch, sacrum, anterior iliac crests, or the C7 vertebral process) prior to the procedure, may be used to provide references for accurate intra-procedure geographic localization (GPS type) of the diseased tissue in question.

Voice activation of cameras, blue and white light LED activation and GPS localization can all be available with the endoscopic devices.

Many solid and liquid substances naturally emit fluorescent radiation when irradiated or illuminated with ultraviolet (UV), visible, or near-infrared (NIR) light. However, the radiation may fall within wide wavelength bands of low intensity. In many cases, observations may be partially obscured by natural fluorescence (auto-fluorescence) emanating simultaneously from many different compounds present in the tissue under examination. Imaging devices such as microscopes, endoscopes and charged couple devices (CCDs), can be fitted with filters for a selected wavelength bands to screen out undesired fluorescence emanating from the object under observation in order to view the desired area of fluorescence.

Both tumors and healthy tissue may fluoresce naturally (auto fluorescence), albeit often at different wavelengths. Consequently, when light-activated (UV, visible or NIR) fluorescence is used to detect tumors against a background of healthy tissue, identification of tumor tissue may be difficult. Unlike most other cells of the body, tumor cells may possess a natural ability to concentrate and retain hematoporphyrin derivative dyes. Based upon this discovery, a technique was developed wherein a hematoporphyrin derivative fluorescent dye is administered and allowed to concentrate in a tumor to be examined to increase the fluorescence from the tumor as compared with that of healthy background tissue. Hematoporphyrin dyes fluoresce within a fluorescence spectrum between 610 and 700 nm, a spectrum easy to detect. However, the natural fluorescence from healthy cells may be much more intense than that from the dyes, and has a broader fluorescence spectrum. Thus, the use of fluorescent dyes in diagnosis of tumors has not been wholly successful. Disclosed herein, the use of fluorescein, fluorescein-type derivatives, and fluorophores with excitation in the 400-510 nm range and emission in the 515-550 range bypasses the problem by providing tumor fluorescence that is bright green and easily distinguished from normal tissue.

In endoscopic systems such as disclosed in U.S. Pat. No. 4,821,117, a body part having abnormal or diseased tissue, such as a cancer, may be identified by comparing an image produced by visible light illumination of the internal organ with the image produced by fluorescence. To aid in visualizing the images received, endoscopic systems can utilize a still or video camera attached to a fiber optic scope having an optical guide fiber for guiding a beam from an external radiation source to the internal organ, and another optical guide fiber for transmitting a fluorescent image of the affected area to a monitor for viewing. Images of the object obtained independently by visible and fluorescent light using a TV camera can be stored in memory, and can be simultaneously displayed in a television monitor to visually distinguish the affected area of the body part from the healthy background tissue.

In another type of procedure, such as is described in U.S. Pat. No. 4,786,813, a beam-splitting system splits the fluorescence radiation passing though the optical system into at least three parts, each of which forms a respective image of the object corresponding to each of the wavelength regions received. A detector produces a cumulative weighted signal for each image point corresponding to a single point on the object. From the weighted signal values of the various points on the object, an image of the object having improved contrast is produced. This technique is used to aid in distinguishing the fluorescence from the affected tissue from that produced by normal tissue.

U.S. Pat. No. 4,719,508 discloses a method utilizing an endoscopic photographing apparatus wherein the endoscope includes an image sensor for successively generating image signals fed to a first frame memory for storing the image signals and a second frame memory for interlacing and storing image signals read successively from the first frame memory. The stored, interlaced image signals are delivered to a TV monitor for display to aid in visualizing the affected body part. Here, the white and blue light generated images are processed as taken and can be streamed wirelessly directing to an external smartphone, tablet, laptop, desktop or television with the need for storing and re-processing images. No CCD capture device is needed. In addition, if the subject being examined does need to have open body cavity surgery at any time, no CCD is needed to continue viewing the diseased body part as the fluorophore-tagged tumor targeting construct can be viewed directly with simple overhead or handheld lighting devices that provide white light and blue LED light (400-510 nm). This direct viewing capability exists throughout the duration of the procedure.

These prior endoscopic systems, which rely on photographic processing of the image of the area of interest (i.e., via a TV monitor), have historically relied on increasingly complex and expensive equipment and substitute image processing to construct a diagnostic image (i.e., indirect viewing) for direct viewing of the affected body part without image processing, as by any type of camera or image processing device. A major shortfall of these prior systems is that they all require specialized operator training and expertise, expensive, complex and technically sophisticated equipment, and may not be generally available in community medical facilities. In addition, these prior systems may increase the time required to complete a surgical procedure, thereby adding to the patient's time under anesthesia, and subsequent risks therefrom. Finally, if the technology fails at any time during the operative procedure, there is no advantage over direct visualization with white light and no fluorescence of the diseased part in question is available.

These prior endoscopic systems not intended to be used with laser light sources and do not involve the use of fluorophores that are taken up by tumor or diseased tissue with the intent of destroying the diseased tissue though the use of fluorophores or compounds that generate heat when excited by laser light. Such methods have two major drawbacks. Disease states other than tumors cannot be diagnosed, and laser visualization must be delayed for a period of two days or more after administration of the fluorescent dye to allow the dye to clear from normal tissue.

Monoclonal antibodies and other tissue and tumor-avid compounds specific for tumors as well as diseased and normal tissues have been developed for use in diagnosis and treatment of tumors and other diseased tissue. Tumor-avid moieties are disproportionately taken up (and, or optionally are metabolized by tumor cells). Several well-known tumor-avid compounds are deoxyglucose, which plays a role in glycolysis in tumor cells; somatostatin, which binds to and/or is taken up by somatostatin receptors in tumor cells and particularly in endocrine tumors; methionine, histidine and folic acid, which can be used as a substrate for metabolism in a wide array of tissues but are taken up preferentially by certain malignant tissues. In such studies, deoxyglucose is used as a radio-tagged moiety, such as fluorodeoxyglucose (18F-deoxyglucose), for detection of tumors of various types. One example would be positron emission tomography (PET) scans. It is believed that tumor cells experience such a mismatch between glucose consumption and glucose delivery that anaerobic glycolysis must be relied upon, thereby elevating the concentration of the radioactive tag in tumor tissue. It is also a possibility that the elevated concentration of deoxyglucose in malignant tumors may be caused by the presence of isoenzymes of hexokinase with abnormal affinities for native glucose or its analogs (A. Gjedde, Chapter 6: “Glucose Metabolism,” Principles of Nuclear Medicine, 2nd Ed., W.B. Saunders Company, Philadelphia, Pa., pages 54-69). Similarly, due to the concentration of methionine and somatostatin in tumor tissue, radio-tagged methionine and somatostatin, and fragments or analogs thereof, can be used in the art for non-invasive imaging of a variety of tumor types. One such procedure is known as somatostatin receptor scintigraphy (SRS).

Scintigraphic techniques have been difficult to apply during a surgical procedure because of the equipment necessary for viewing the image provided by the radioisotope. This obstacle can be been overcome with systems such as the NeoProbe® and LymphoSeek® systems. However, at the time that the surgeon has made the incision or entered the body cavity it could be useful to “see” the outlines of the diseased tissue in real time without the need for time-consuming, expensive image processing equipment. In addition, even using the best surgical techniques, it is well known that residual microscopic clusters of cells can and frequently are left behind after surgical excision of malignant tissue. Scintigraphic technology as described herein can be used adjunctively for the localization and detection of diseased tissue and can provide an advantage to the use of tumor fluorescence, when tumor tissue might be below the surface of the tissues examined and might not readily be see with the blue excitation light (400-510 nm). In addition, the radioisotopes attached to the tumor-targeting constructs allow for the pre-operative nuclear scanning to provide additional reference information on the location of tumor tissue prior to examination of the tissue using white and blue light illumination. It could also allow for treatment of diseased tissue if a dual emitting (gamma and beta) emitters were used.

Fiberoptic endoscopic devices with light sources that provide white as well as blue light (400-510 nm) can be utilized to visualize a broad range of putative disease sites without the need for use of image processing equipment. Where real-time visualization is by means of endoscopic devices (flexible or rigid, or capsule), and robotic devices, direct visualization (as opposed to images created by image processing equipment) offers the additional advantage that the equipment required is comparatively simple to use, is not prone to malfunction, and is less expensive than the equipment required to process images or create photographic displays from such images and no additional time is spent in image processing. In addition, there is a need in the art for a method of identifying diseased or abnormal tissue during surgical procedures so that immediate resection or biopsy of the identified tissue can be performed while the surgeon “sees” the outlines of the diseased or abnormal tissue.

Fiberoptic and rigid endoscopes as well as capsule endoscopes can be utilized for a variety of procedures including colonoscopy, upper gastrointestinal endoscopy, bronchoscopy, thorascopy, angioscopy, culposcopy, cystoscopy, laryngoscopy, cisternal endoscopy, arthroscopy, and laparoscopy. Fiberoptic endoscopy can provide real time accurate visualization of internal body parts and can use white light from a light source external to the body that passes through a bundle of glass fibers to illuminate the internal organ and a second bundle of fibers to visualize the internal organ being visualized (see diagram). This same fiberoptic and rigid endoscopic equipment can be used for visualizing fluorescent-tagged diseased tissue during endoscopy or robotic surgery, when the visual field is illuminated with blue (400-510 nm) excitation light and a filter (515 nm) over the viewing device is used to filter out the blue excitation light and allow visualization of the fluorescent emission light (green fluorescence in our examples).

Endoscopic systems can utilize fiberoptics to provide a means of delivering light (through a fiberoptic bundle) and to provide a means of visualizing the internal organ (through a separate fiberoptic bundle for viewing). Described herein, the endoscopy does not utilize fiberoptics but instead utilizes cameras mounted at the distal (internal) viewing end of the endoscope or capsule endoscope. Light is provided by high intensity micro-LEDs for illumination at the distal viewing end of the endoscopes (white LEDs for normal visualization and blue LEDs (400-510 nm) for visualization of fluorophore-tagged diseased tissue). While near-Infrared light (NIR) sources could also be used they would require the use of a capture device (i.e. CCDs). The LED light sources at the distal viewing end of the endoscopes require minimal energy for bright illumination and can be run on simple external batteries. The cladding cover of the endoscope can be used to protect the wires connecting the camera to the external viewing device, markedly reducing the weight of glass fiberoptics, decreasing the cost, and simplifying the technology.

Viewing the internal organ and any diseased tissue at the distal end of the endoscope can be through 1 or more micro-cameras mounted at the distal viewing end of the microscope (these cameras could be digital cameras, similar to cameras found in smart phones or could also be from a Nano-eye® camera). One camera can be used to view the internal organ with white light and the 2nd camera can be used to view the internal organ when using the blue LED (400-510 nm) lights. The camera for viewing with blue light (400-510 nm) can have a yellow filter (515 nm) over the camera lens to eliminate the blue excitation light allowing clear visualization of the emission light from the fluorophore-tagged construct bound to the diseased tissue to be identified. Additional cameras could also be used for imaging in 3-D (two with white LED illumination) and one with blue LED (400-510 nm) illumination.

The viewing cameras can be connected through wires in the endoscope cladding or could be connected wirelessly to a viewing device located external to the subject being examined. The external viewing and image capture device utilized can be a simple smart phone (i.e. an iPhone, Android or Google phone, etc.); a tablet device (i.e an iPad, Samsung tablet, Kindle, etc.), a laptop or desktop computer or television monitor.

The external viewing device can be connected wirelessly via wi-fi connection, with transmission of images to a distant site being by phone connection, or satellite connection for real-time streaming of the imaging process and images.

Wireless localization devices could be placed at locations on the body prior to the procedure to provide reference for geographic localization (GPS type) of the diseased tissue in question. These locations could include the anterior iliac crests, the posterior iliac crests, the sacrum, coccyx, pubic ramus, sternal notch, C7 cervical spine etc.

The endoscopic device can be fitted for manipulation and navigation within the internal organs with mechanisms currently used in fiberoptic endoscopes. External operating controls can be similar to the operating controls commonly found in fiberoptic endoscopes (Olympus, Storz, Fuji Pentax, Stryker).

The devices and methods described herein can be used with fluorophore-tagged monoclonal antibodies (MAbs) or fluorophore-tagged tissue avid compounds (TACs) (see G. Luiken patents) and overcomes many of these problems in the art of endoscopy and tumor imaging by providing simple, battery-operated, low cost endoscopic method(s) for the in vivo identification of diseased tissue in a subject in need thereof. As such, described herein are endoscopic methods for visually detecting tumor tissue, diseased tissue, or normal tissue at an interior or exterior body site using tumor-specific or tissue specific fluorescent targeting constructs, which are excited by light in the visible range (i.e. 400-510 (preferably 470-495 nm), to allow more accurate identification and potentially removal of all such localized tissue, and for identification of this fluorescent-tagged tissue with distal end camera viewing rigid, flexible, capsule or robotic endoscopes without the need for fiberoptics and without the need image capture devices (i.e. CCDs) and with image transmission through smart phones, tablet devices, or similar image capture devices, through wire connections or wirelessly.

In an embodiment, the method includes illuminating an in vivo body part of the subject containing tumor or diseased tissue or normal tissue with light having at least one excitation wavelength in the range from 400 nm to about 510 nm. Fluorescent targeting constructs can be previously injected into the subject and can be bound to and/or been taken up by the tumor or diseased tissue in the body part being examined. Diseased tissue can be identified by viewing the fluorescence emanating from the fluorescent targeting constructs.

The fluorescent targeting construct may comprise a fluorophore-tagged antibody (partial antibody, Fab fragment, diabody) or fluorophore-tagged tumor avid moiety or fluorophore-tagged tissue compound, linked to albumin and such constructs may also be tagged with a radio-isotope (such radio-isotope being a dual emitting isotope and capable of therapeutic potential as well as being detectable through external nuclear imaging and internal (endoscopic) detection. The fluorophore-tagged antibody or fluorophore-tagged tumor avid moiety is responsive to the excitation wavelength administered to the subject through the use of LEDs (400-510 nm), and the radio-isotope is capable of being detected by an external radiation scanner (i.e. PET scan), radiation detection device mounted in the distal viewing or detection end of a rigid, flexible, capsule or robotic endoscopic device.

In another embodiment, described herein are methods for utilizing a diagnostic procedure during surgery in a subject in need thereof. In this embodiment s, an in vivo body part (e.g., tissue or organ) of the subject containing diseased tissue can be illuminated with light having at least one excitation wavelength in the range from about 400-510 nm. The targeting construct can be pre-administered to the subject and can be specifically bound to and/or taken up by the diseased tissue or organ in the body part. The targeting construct fluoresces in response to the at least one excitation wavelength and can be directly viewed to determine the location and/or surface area of the diseased tissue in the subject. Because the fluorescence can be directly viewed through the endoscope and can be limited to the diseased tissue, all or at least a portion of the diseased tissue can be removed. The targeting construct comprises a fluorophore-tagged antibody or fluorophore-tagged tumor avid moiety.

In addition, in one embodiment, the fluorophore-tagged tumor avid moiety may additionally have a radioisotope (with dual energy emission for scanning detection as well as for therapy) attached. The utility of combining a radio-isotope to a fluorophore-tagged tumor targeting construct allows additional detection through the use of radio-isotope detected devices as well as providing “adjuvant” radiation therapy to small distant microscopic metastatic cancer cells, not removed at the time of the primary surgery done with tumor fluorescence. In essence, the bulk of the primary tumor can be removed using induced tumor fluorescence (using fluorophore-tagged MAbs or fluorophore-tagged tumor-avid compounds (TACs). Microscopic metastases can be destroyed by the radio-isotope labeled and fluorophore-tagged MAbs at distant sites within the body.

In another embodiment, the digital endoscopes can have embedded in the distal viewing end of the scope a small Geiger counter that could be connected through a cable in the endoscope cladding or can transmit data wirelessly to an external source.

Described herein are endoscopic devices for the in vivo identification, and surgical therapy of diseased tissue in a subject in need thereof. The devices can include means for illuminating an in vivo body part of the subject containing diseased tissue with light having at least one excitation wavelength in the range from about 400-510 nm. The endoscopic devices can be used to visualize fluorescence emanating from diseased tissue within the body. The diseased tissue has attached a fluorescent targeting construct that can be administered (generally intravenously) to the subject and which can be bound to and/or taken up by the diseased tissue in the body part.

Light for illumination can emanate from very small white and micro-white LEDs and blue LED (400-510 nm) located at the distal end of the endoscopes Therefore, the excitation light can contain at least one wavelength of light that illuminates surrounding tissue as well as excites fluorescence from the fluorescent targeting construct. The excitation light may be monochromatic or polychromatic.

In one embodiment, two or four viewing cameras at the distal end of the endoscope can be used to view the organ being examined. One camera (without filter) can be used to view the organ when examined with white light illumination, and the 2^{nd} camera (with yellow (515 nm) filter can be used to view the organ being examined with blue LED light (400-510 nm) illumination. To compensate for the tendency of the normal tissue background to be seen as blue and to obscure the desired visualization of the fluorophore targeting construct, a yellow filter (515 nm) can be used to screen out wavelengths below about 515 nm in the excitation light, thereby eliminating the blue excitation wavelengths. Use of a filter is encompassed by the term “directly viewing” as applied to the methods described herein. Use of one or more filters to screen out wavelengths of light in a selected wavelength band or screen out wavelengths except those the desired wavelength band is well known in the art. In addition the use of additional cameras could provide the capability to view the diseased or tumor tissue in 3D with white and blue light (400-510 nm).

Light in the 401 nm to 510 nm wavelength range is readily absorbed in tissue. Accordingly, the diseased tissue (and bound targeting construct) can be “exposed” to the excitation light by endoscopic delivery of the light to an interior location. The methods described herein are particularly suited to in vivo detection of diseased tissue located at an interior site in the subject, such as within a natural body cavity, hollow organ or a surgically created opening, where the diseased tissue is “in plain view” (i.e., exposed to the human eye) to facilitate a procedure of biopsy or surgical excision. As the precise location and/or surface area of the tumor tissue can be determined by the diagnostic procedure described herein, the methods described herein are valuable guides to the surgeon, who needs to “see” in real time the outlines, size, etc., of the diseased tissue or mass to be resected as the surgery proceeds.

If the putative diseased site is a natural body cavity or surgically produced interior site, an endoscopic device can be used to deliver the excitation light to the site, to receive fluorescence emanating from the site within a body cavity, and to aid the visualization of the fluorescence emanating from the diseased tissue. For example, the camera in the distal end of the endoscopic device can be used to focus on the detected fluorescence. As used herein, such endoscopically-visualized fluorescence is said to be “directly viewed” by the practitioner and the tissue or organ to which the targeting construct binds or in which it is taken up must be “in plain view” to the endoscope since the light used may not contain wavelengths of light that require an image capture device (i.e. CCD) as needed in the near infrared range. Alternatively, as described above, the excitation light may be delivered by any convenient means, such as a hand-held LED or fixed light source, into a body cavity or surgical opening containing a targeting construct administered as described herein and the fluorescent image so produced can be directly visualized by the eye of the observer through the camera at the distal end of the endoscope. The fluorescence produced by the methods described herein is such that it can be viewed without aid of an image processing device, such as a CCD camera (since near-infrared light is not used), photon collecting device, and the like if that becomes necessary at any time during the procedure undertaken (i.e. colonoscopy, colposcopy, cystoscopy, gastroscopy, thoracoscopy, etc.)

In one embodiment, diseased or abnormal tissues or organs can be contemporaneously viewed through a surgical opening to facilitate a procedure of biopsy or surgical excision. As the location and/or surface area of the diseased tissue or organ are readily determined by the diagnostic procedures described herein, the methods are valuable guides to the surgeon, who needs to know the exact outlines, size, etc., of the mass, for example, for resection as the surgery proceeds.

Accordingly, this embodiment includes methods for utilizing a diagnostic procedure during surgery in a subject in need thereof by illuminating an in vivo body part of the subject containing diseased tissue with light having at least one excitation wavelength in the range from about 400-510 nm, directly viewing through the camera, the fluorescence emanating from a targeting construct administered to the subject that has specifically bound to and/or been taken up by the diseased tissue in the body part, wherein the targeting construct fluoresces in response to the at least one excitation wavelength, determining the location and/or surface area of the diseased tissue in the subject, and removing all or at least a portion of the tumor tissue.

In one embodiment, a single type of fluorescent moiety is relied upon for generating fluorescence emanating from the irradiated body part (i.e., from the fluorescent targeting construct that binds to or is taken up by diseased tissue). Since certain types of healthy tissue fluoresce naturally, in such a case it is important to select a fluorescent moiety for the targeting construct that has a predominant excitation wavelength that does not contain sufficient wavelengths in the visible range of light to make visible the surrounding healthy tissue and thus inhibit resolution of the diseased tissue. Therefore, the light source used in this embodiment can emit light in the range from about 400-510 nm. Thus, the methods described herein may involve contact of diseased tissue with a fluorescent targeting construct.

Exemplary fluorescent targeting constructs include anti-tumor antigen antibodies (e.g., FAB fragment, bispecific antibodies, diabodies, or antibody fragments) or tumor avid compounds (e.g. deoxyglucose, methionine, somatostatin, folic acid, hormones, hormone receptor ligands) and a biologically compatible fluorescing moiety. As used herein, the terms “fluorophore-tagged antibody” and “fluorophore-tagged tumor avid compound” respectively refer to such fluorescent targeting constructs that are responsive to specific excitation wavelengths administered to a subject in need thereof. The binding of fluorophores to the targeting molecules can be through well described linkers well known to those skilled in the art.

The fluorescing moiety of the targeting construct can be any chemical or protein moiety that is biologically compatible (e.g., suitable for in vivo administration) and which fluoresces in response to excitation light as described herein. Since the targeting ligand is administered to living tissue, biological compatibility includes the lack of substantial toxic effect to the individual in general if administered systemically, or to the target tissue, if administered locally, at the dosage administered. Non limiting examples of fluorophores that can be used include fluorescein, fluorescein derivatives, tetracycline, quinine, mithramycin, Oregon green, and cascade blue, and the like, and combinations of two or more thereof. Molecules with similar excitation and emission spectra and with similar safety profiles may be used as they are developed.

Additional non-limiting examples of fluorescent compounds that fluoresce in response to an excitation wavelength in the range from 400-510 nm are found in Table 1 below:

TABLE 1

| | Excitation | Emission |
|---|---|---|
| Compound | Range (nm) | Range (nm) |
| Acridine Red | 455-600 | 560-680 |
| Acridine Yellow | 470 | 550 |
| Acriflavin | 436 | 520 |
| AFA (Acriflavin Feulgen SITSA) | 355-425 | 460 |
| Alexa Fluor | 470-490 | 520 |
| ACMA | 430 | 474 |
| Astrazon Orange | 470 | 540 |
| Astrazon Yellow | 450 | 480 |
| Atabrine | 436 | 490 |
| Auramine | 460 | 550 |
| Aurophosphine | 450-490 | 515 |
| Aurophosphine G | 450 | 580 |
| Berberine Sulphate | 430 | 550 |
| BOBO-1, BO-PRO-1 | 462 | 481 |
| BOPRO1 | 462 | 481 |
| Brilliant Sulpho-flavin FF | 430 | 520 |
| Calcein | 494 | 517 |
| Calcofluor White | 440 | 500-520 |
| Cascade Blue | 400 | 425 |
| Catecholamine | 410 | 470 |
| Chinacrine | 450-490 | 515 |
| Coriphosphine O | 460 | 575 |
| DiA | 456 | 590 |
| Di-8-ANEPPS | 488 | 605 |
| DiO [DiOC18(3)] | 484 | 501 |
| Diphenyl Brilliant Flavine 7GFF | 430 | 520 |
| Euchrysin | 430 | 540 |
| Fluorescein | 494 | 518 |
| Fluorescein Iso-thiocyanate (FITC) | 490 | 525 |
| Fluo 3 | 485 | 503 |
| FM1-43 | 479 | 598 |
| Fura Red (low[Ca2+]) | 472 | 657 |
| Fura Red (high[Ca2+]) | 436 | 637 |
| Genacryl Brilliant Yellow 10GF | 430 | 485 |
| Genacryl Pink 3G | 470 | 583 |
| Genacryl Yellow SGF | 430 | 475 |
| Gloxalic Acid | 405 | 460 |
| 3-Hydroxypyrene-5,-8,10-TriSulfonic Acid | 403 | 513 |
| 7-Hydroxy-4-methylcourmarin | 360 | 455 |
| 5-Hydroxy-Tryptamine (5-HT) | 380-415 | 520-530 |
| Lucifer Yellow CH | 425 | 528 |
| Lucifer Yellow VS | 430 | 535 |
| LysoSensor Green DND-153, DND-189 | 442 | 505 |
| Maxilon Brilliant Flavin 10 GFF | 450 | 495 |
| Maxilon Brilliant Flavin 8 GFF | 460 | 495 |
| Mitotracker Green FM | 490 | 516 |
| Mithramycin | 450 | 570 |
| NBD | 465 | 535 |
| NBD Amine | 450 | 530 |
| Nitrobenzoxadidole | 460-470 | 510-650 |
| Nylosan Brilliant Flavin E8G | 460 | 510 |
| Oregon Green 488 fluorophore | 496 | 524 |
| Phosphine 3R | 465 | 565 |
| Quinacrine Mustard | 423 | 503 |
| Rhodamine 110 | 496 | 520 |
| Rhodamine 5 GLD | 470 | 565 |
| Rhodol Green fluorophore | 499 | 525 |
| Sevron Orange | 440 | 530 |
| Sevron Yellow L | 430 | 490 |
| SITS (Primuline) | 395-425 | 450 |
| Sulpho Rhodamine G Extra | 470 | 570 |
| SYTO Green fluorescent nucleic acid stains | 494 | 515 |
| Thioflavin S | 430 | 550 |
| Thioflavin 5 | 430 | 550 |
| Thiozol Orange | 453 | 480 |
| Uranine B | 420 | 520 |
| YOYO-1, YOYO-PRO-1 | 491 | 509 |

Since the fluorescence properties of biologically compatible fluorophores are well known, or can be readily determined by those of skill in the art, the skilled practitioner can readily select a useful fluorophore or useful combination of fluorophores, and match the wavelength(s) of the excitation light to the fluorophore(s). The toxicity of fluorescein is minimal as it has been used safely in vivo in humans for many years, but the toxicity of additional useful fluorophores can be determined using animal studies as known in the art.

The targeting construct (e.g., the ligand moiety of the targeting construct) can be selected to bind to and/or be taken up specifically by the target tissue of interest, for example to an antigen or other surface feature contained on or within a cell that characterizes a disease or abnormal state in the target tissue. As in other diagnostic assays, it may be desirable for the targeting construct to bind to or be taken up by the target tissue selectively or to an antigen associated with the disease or abnormal state; however, targeting constructs containing ligand moieties that also bind to or are taken up by healthy tissue or cell structures can be used if the concentration of the antigen in the target tissue or the affinity of the targeting construct for the target tissue is sufficiently greater than for healthy tissue in the field of vision so that a fluorescent image representing the target tissue can be clearly visualized as distinct from any fluorescence coming from healthy tissue or structures in the field of vision. For example, colon cancer is often characterized by the presence of carcinoembryonic antigen (CEA), yet this antigen is also associated with certain tissues in healthy individuals. However, the concentration of CEA in cancerous colon tissue is typically greater than is found in healthy tissue, so an anti-CEA antibody could be used as a ligand moiety. In another example, deoxyglucose is taken up and utilized by healthy tissue to varying degrees, yet its metabolism in healthy tissues, except for certain known organs, such as the heart, is substantially lower than in tumor tissue. A large number of tumor directed MAbs are well described including anti-CA15-3, CA19-9, CEACAM6, EpCam, FOLR1, MAGE, CA125, PSMA, TTF1, VEGF, HER2, HER3, etc. to name a few and many additional are developed each year. The known pattern of deoxyglucose consumption in the body can therefore be used to aid in determination of those areas wherein unexpectedly high uptake of deoxyglucose signals the presence of tumor cells. Wireless localization devices could be placed at locations on the body prior to the procedure to provide reference for geographic localization (GPS type) of the diseased tissue in question. These locations could include the anterior iliac crests, the posterior iliac crests, the sacrum, coccyx, pubic ramus, sternal notch, C7 cervical spine etc. As another illustrative example, breast cancer is characterized by the production of cancerous tissue identified by monoclonal antibodies to CA15-3, CA19-9, CEA, or HER2/neu. It is contemplated that the target tissue may be characterized by cells that produce either a surface antigen for which a binding ligand is known, or an intracellular marker (i.e. antigen), since many targeting constructs penetrate the cell membrane. Representative disease states that can be identified methods described herein include such various conditions as different types of tumors, bacterial, fungal and viral infections, and the like. As used herein “abnormal tissue” includes precancerous conditions, cancer, necrotic or ischemic tissue, and tissue associated with connective tissue diseases, and auto-immune disorders, and the like. Further, examples of the types of target tissue suitable for diagnosis or examination methods described herein include cancer of breast, lung, colon, prostate, pancreas, skin, stomach, small intestine, testicle, head and neck, thyroid, gall bladder, brain, endocrine tissue, and the like, as well as combinations of any two or more thereof.

Representative examples of antigens for some common malignancies and the body locations in which they are commonly found are shown in Table 2 below. Targeting ligands, such as antibodies, for these antigens are known in the art.

TABLE 2

| Tumor Antigen | Location or Cancer Type |
|---|---|
| CEA (carcinoembryonic antigen) | Colon, breast, lung, pancreas, head |
| | and neck, medullary thyroid |
| CEACAM6 | Pancreas, colon, breast, stomach, |
| | esophagus |
| PSA (prostate specific antigen) | Prostate cancer |
| PSMA (prostate specific membrane | Prostate cancer |
| antigen) | |
| CA-125 | Ovarian cancer, breast, colon, lung |
| CA 15-3 | Breast cancer, lung, colon, pancreas, |
| CA 19-9 | Pancreas cancer |
| HER2/neu | Breast cancer |
| TTF1 | Lung cancer |
| α-feto protein | Testicular cancer, hepatic cancer |
| β-HCG | Testicular cancer, choriocarcinoma |
| MUC-1 | Breast cancer, colon, lung, |
| MUC-2 | Colorectal cancer, colon, lung |
| TAG 72 | Breast cancer, colon cancer, and |
| | pancreatic cancer |
| Estrogen receptor | Breast cancer, uterine cancer |
| Progesterone receptor | Breast cancer, uterine cancer |
| AR (androgen receptor) | Prostate cancer |
| EGFr (epidermal growth factor | Bladder cancer |
| receptor) | |
| IGFr (insulin like growth factor) | Sarcoma |

In one embodiment, the ligand moiety of the targeting construct can be a protein or polypeptide, such as an antibody, or biologically active fragment thereof, preferably a monoclonal antibody. The supplemental fluorescing targeting construct(s) may also be or comprise polyclonal or monoclonal antibodies tagged with a fluorophore. The term “antibody” as used herein includes intact molecules as well as functional fragments thereof, such as Fab, F(ab′)2, and Fv that are capable of binding the epitopic determinant. These functional antibody fragments retain some ability to selectively bind with their respective antigen or receptor and are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; (Fab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule. (6) Diabody; (7) Polyfunctional antibody.

Methods of making these fragments are known in the art. (See for example, Harlow & Lane, Antibodies: A Laboratory Manual, Cold Spring Harbor Laboratory, New York, 1988).

A variety of methods are available for the production of monoclonal antibodies (see Of mice and men: hybridoma and recombinant antibodies. Immunol Today, Little M, Kipriyanov S M, Le Gall F, Moldenhauer G., August; 21 (8): 364-70, 2000), and include the production of fully human monoclonal antibodies from rabbit hybridomas, for example in Pytela, et al., U.S. Pat. No. 7,429,487, and U.S. Pat. No. 8,062,867.

In an embodiment, the ligand moiety in the fluorescent targeting construct can be selected from among the many biologically compatible tumor-avid moieties that bind with specificity to receptors and/or are preferentially taken up by tumor cells, and can be used as the ligand moiety in targeting constructs. Tumor-avid moieties that can “taken up” by tumor cells may enter the cells through surface or nuclear receptors (e.g., hormone receptors), pores, hydrophilic “windows” in the cell lipid bilayer, and the like.

Illustrative of this class of tumor-avid moieties are somatostatin, somatostatin receptor-binding peptides, deoxyglucose, methionine, histidine, folic acid, and the like.

Additional examples of biologically compatible tumor-avid compounds that bind with specificity to tumor receptors and/or are preferentially taken up by tumor cells include mammalian hormones, particularly sex hormones, neurotransmitters, and compounds expressed by tumor cells to communicate with each other that are preferentially taken up by tumor cells, such as novel secreted protein constructs arising from chromosomal aberrations, such as transfers or inversions within the clone.

The fluorescent moiety sensitive to an excitation wavelength in the 400-510 nm range can be linked to the tumor-avid compound used as the ligand moiety in the targeting construct by any method presently known in the art for attaching two moieties, if the attachment of the linker moiety to the ligand moiety does not substantially impede binding of the targeting construct to the target tissue and/or uptake by the tumor cells, for example, to a receptor on a cell. Those of skill in the art will know how to select a ligand/linker pair that meets this requirement (L. J. Hofland et al., Proc. Assoc. Am. Physicians 111:63-9, 1999).

The targeting constructs and supplemental targeting constructs can be administered by any route known to those of skill in the art, such as intravenously, intraarticularly, intracisternally, intraocularly, intraventricularly, intrathecally, intramuscularly, intraperitoneally, intradermally, intracavitarily, and the like, as well as by any combination of any two or more thereof.

The targeting construct can be administered in a “diagnostically effective amount.” As used herein, a “diagnostically effective amount” refers to the quantity of a targeting construct necessary to aid in direct visualization of any target tissue located in the body part under investigation in a subject. As used herein, the term “subject” refers to any mammal, such as a domesticated pet, farm animal, or zoo animal, but preferably is a human. Amounts effective for diagnostic use will, of course, depend on the size and location of the body part to be investigated, the affinity of the targeting construct for the target tissue, the type of target tissue, as well as the route of administration.

What is claimed is:

1. An endoscopic device, comprising:
at least one white light source;
at least one blue light source which emits light with a wavelength between 400 nm and 510 nm;
a first camera; and
a first filter capable of filtering light with a wavelength less than 515 nm.

2. The endoscopic device of claim 1, wherein the endoscopic device is a flexible or rigid endoscope.

3. The endoscopic device of claim 1, wherein the endoscopic device is a capsule endoscope.

4. The endoscopic device of claim 1, wherein the blue light source emits light with a wavelength between 470 nm and 495 nm.

5. The endoscopic device of claim 1, further comprising a second camera, wherein the first filter is positioned over the second camera such that light with a wavelength of less than 515 nm is filtered from the view of the second camera.

6. The endoscopic device of claim 5, further comprising a third camera, a fourth camera, and a second filter positioned over the fourth camera such that light with a wavelength of less than 515 nm is filtered from the view of the fourth camera, wherein the first camera and the third camera are capable of producing a three dimensional image when used together, and the second camera and the fourth camera are capable of producing a three dimensional image when used together.

7. The endoscopic device of claim 1, further comprising a radiation detection device which is located at the distal end of the endoscopic device.

8. The endoscopic device of claim 6, wherein the radiation detection device is a miniature Geiger counter.

9. The endoscopic device of claim 1, further comprising a geographic localization guidance chip.

10. A method of detecting diseased tissue of a subject in need thereof, comprising:
administering a diagnostically effective amount of a targeting construct to a subject, wherein the targeting construct is capable of specifically binding to and/or being taken up by the diseased tissue of the subject;
illuminating a body part of the subject with light having at least one excitation wavelength in the range from about 400 to about 510 nm, wherein the targeting construct fluoresces in response to the at least one excitation wavelength;
viewing fluorescence emanating from the targeting construct through a first camera;
determining the location and/or surface area of the diseased tissue.

11. The method of claim 10, wherein the targeting construct is selected from the group consisting of an anti-tumor antigen antibody, a tumor avid compound, and a biologically compatible fluorescing moiety.

12. The method of claim 11, wherein the targeting construct is an anti-tumor antigen antibody selected from the group consisting of a FAB fragment, a bispecific antibody, a diabody, and an antibody fragment.

13. The method of claim 11, wherein the targeting construct is a tumor avid compound selected from the group consisting of deoxyglucose, methionine, somatostatin, folic acid, a hormone, and a hormone receptor ligand.

14. The method of claim 10, wherein the targeting construct is selected from the group consisting of fluorescein, fluorescein iso-thiocyanate, and Oregon Green 488 fluorophore.

15. The method of claim 10, wherein the diseased tissue is cancerous tissue.

16. The method of claim 15, wherein the cancerous tissue is cancerous tissue from colon cancer.

17. The method of claim 15, wherein the cancerous tissue is cancerous tissue from breast cancer.

18. The method of claim 10, wherein a filter is positioned over the first camera and the filter is capable of filtering light with a wavelength of less than 515 nm.

19. The method of claim 10, further comprising viewing the body part through a second camera.

20. The method of claim 10, further comprising observing the geographic location of the diseased tissue using a geographic localization guidance chip.
